(19) United States
(12) Patent Application Publication
(10) Pub. No.: US 2012/0051505 A1
(43) Pub. Date: Mar. 1, 2012

(54) RADIATION IMAGE PROCESSING DEVICE, RADIATION IMAGE PROCESSING METHOD AND RADIATION IMAGE PROCESSING PROGRAM STORAGE MEDIUM

(75) Inventors: Yasunori OHTA, Ashigarakami-gun (JP); Yusuke KITAGAWA, Ashigarakami-gun (JP); Tetsuro KUSUNOKI, Ashigarakami-gun (JP); Naoyuki NISHINO, Ashigarakami-gun (JP); Noriaki IDA, Ashigarakami-gun (JP); Takeshi KAMIYA, Ashigarakami-gun (JP)

(73) Assignee: FUJIFILM CORPORATION, Tokyo (JP)

(21) Appl. No.: 13/220,823

(22) Filed: Aug. 30, 2011

(30) Foreign Application Priority Data
Aug. 31, 2010 (JP) 2010-194391

(51) Int. Cl. A61B 6/02

(52) U.S. Cl. 378/41

(57) ABSTRACT

Disclosed is a radiation image processing device that includes a storage unit; and a control unit that performs a control operation to associate a plurality of images of an object captured with radiation from different angles, with information about stereoscopic viewing conditions under which stereoscopic viewing has been performed with the plurality of images, the plurality of images associated with the information being stored into the storage unit through the control operation.

### CROSS-REFERENCE TO RELATED APPLICATION

This application claims priority under 35 USC 119 from Japanese Patent Application No. 2010-194391 filed on Aug. 31, 2010, the disclosure of which is incorporated by reference herein.

### BACKGROUND OF THE INVENTION

1. Field of the Invention

The present invention relates to a radiation image processing device, a radiation image processing method and a storage medium, and more particularly, to a radiation image processing device and a radiation image processing method for storing stereoscopically captured radiation images, and a storage medium

2. Related Art

Japanese Patent Application Laid-Open (JP-A) No. 7-227390 discloses an X-ray rotational stereoscopic image capturing apparatus that includes an angle detecting storage unit that adds image capturing angle information to the data about each of plural images, and stores the image data and the information.

However, JP-A No. 7-227390 merely relates to an X-ray rotational stereoscopic image capturing apparatus, and neither concerns stereoscopic image capturing nor discloses storage of information about stereoscopic viewing.

When two radiation images captured from different angles are stereoscopically viewed, how the two radiation images appear to the observer depends on viewing conditions such as a stereoscopic viewing technique and the amount of offset between the two radiation images being stereoscopically viewed. Therefore, the information about the radiation source and the radiation detector used in capturing the images does not guarantee reproducibility for the next viewing. For example, poor reproducibility results in a problem in medical follow-ups. In some cases, the same images are shared between hospitals these days. However, if physicians view the images in different viewing manners and diagnose a symptom based on the differently-viewed images without noticing, different diagnoses are made about the same symptom.

A main object of the present invention is to provide a radiation image processing device and a radiation image processing method that enable stereoscopic viewing of stereoscopically-captured images with high reproducibility.

### SUMMARY

According to a first aspect of the present invention, there is provided a radiation image processing device comprising:

a storage unit; and

a control unit that performs a control operation to associate a plurality of images of an object captured with radiation from different angles, with information about stereoscopic viewing conditions under which stereoscopic viewing has been performed with the plurality of images, the plurality of images associated with the information being stored into the storage unit through the control operation.

According to a second aspect of the present invention, there is provided a radiation image processing method comprising storing a plurality of images of an object captured with radiation from different angles into a storage unit under a control of a control unit, the plurality of images being associated with information about stereoscopic viewing conditions under which stereoscopic viewing has been performed with the plurality of images.

According to a third aspect of the present invention, there is provided a non-transitory computer-readable medium storing a program that causes a computer to perform a process including storing a plurality of images of an object captured with radiation from different angles into a storage unit under a control of a control unit, the plurality of images being associated with information about stereoscopic viewing conditions under which stereoscopic viewing has been performed with the plurality of images.

### BRIEF DESCRIPTION OF THE DRAWINGS

Exemplary embodiments of the present invention will be described in detail based on the following figures, wherein:

FIG. 1 is a schematic view for explaining a radiation image capturing apparatus according to a preferred exemplary embodiment of the invention;

FIG. 2 is a schematic block diagram for explaining the radiation image capturing apparatus according to the preferred exemplary embodiment of the invention;

FIG. 3 is a perspective view for explaining the structure of a stereo display device of the radiation image capturing apparatus according to the preferred exemplary embodiment of the invention;

FIG. 4 is a diagram for explaining a case in which an image on the stereo display device of the radiation image capturing apparatus is stereoscopically viewed according to the preferred exemplary embodiment of the invention;

FIG. 5 is a schematic view for explaining stereo image capturing using the radiation image capturing apparatus according to the preferred exemplary embodiment of the invention; and

FIG. 6 is a schematic view for explaining stereo image capturing using the radiation image capturing apparatus according to the preferred exemplary embodiment of the invention.

### DETAILED DESCRIPTION

The following is a description of a preferred exemplary embodiment of the invention, with reference to the accompanying drawings.

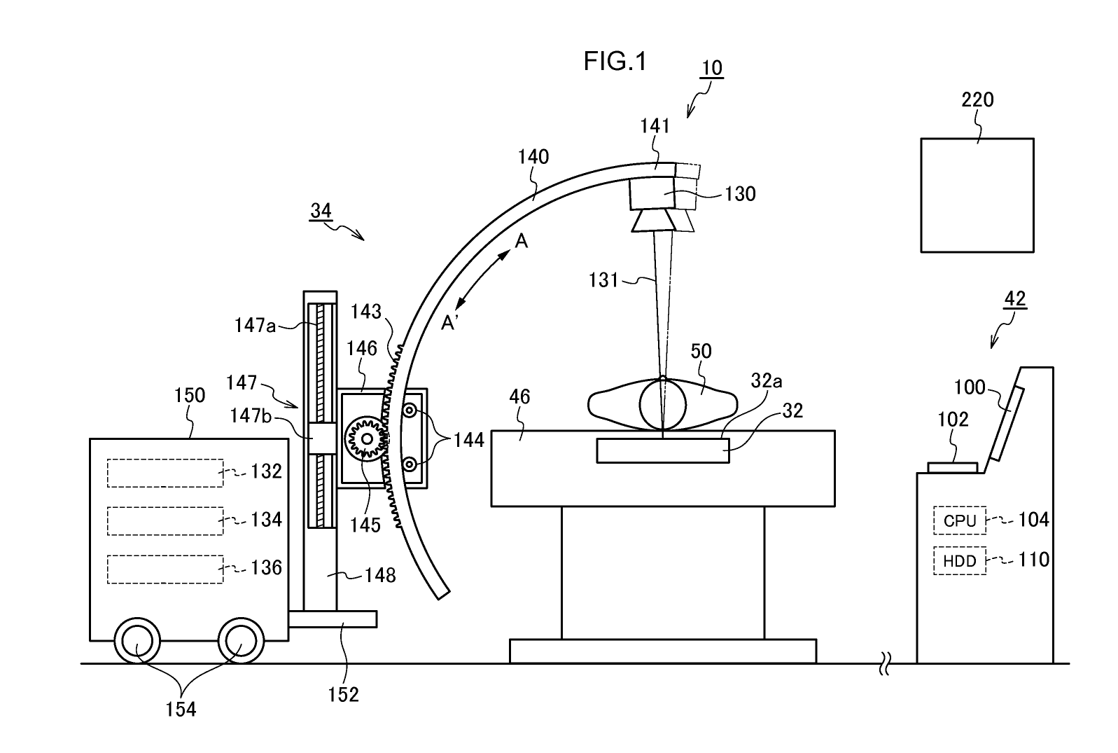

Referring to FIG. 1, a radiation image capturing apparatus 10 of the preferred exemplary embodiment of the invention includes a radiation generator 34, a console 42, a portable radiation image detecting device (hereinafter referred to as the “electronic cassette”) 32, and a stereo display device 220.

The electronic cassette 32 is positioned at a distance from a radiation source 130 of the radiation generator 34 that generates a radiation ray such as an X-ray when a radiation image is captured. In this exemplary embodiment, the electronic cassette 32 is horizontally positioned below an object 50 lying on his/her back on a bed 46, with a distance being kept between the electronic cassette 32 and the object 50. The object 50 is located between the radiation source 130 of the radiation generator 34 and the electronic cassette 32. When a radiation image capturing instruction is issued from the console 42, the radiation source 130 emits an X-ray 131 of a radiation level in accordance with predetermined imaging conditions and the like. The X-ray 131 emitted from the radiation source 130 carries image information after transmitted through the object 50, and then reaches the electronic cassette 32.

The radiation generator 34 includes a main body 150 and a C-shaped arm 140. The radiation source 130 that emits the X-ray 131 is attached to one end 141 of the C-shaped arm 140.

The C-shaped arm 140 is provided to penetrate through a box 146. A gear 143 is formed on an outer circumferential surface of a cylindrical face of the C-shaped arm 140. Rollers 144 attached to the box 146 are in contact with an inner circumferential surface of the cylindrical surface of the C-shaped arm 140. A gear 145 attached to the box 146 meshes with the gear 143 of the C-shaped arm 140. As the gear 145 is rotated by a motor (not shown), the C-shaped arm 140 rotationally moves in a clockwise direction A and a counterclockwise direction A′ shown in the drawing. With this arrangement, the radiation source 130 attached to the C-shaped arm 140 rotationally moves in the clockwise direction A and the counterclockwise direction A′.

As the radiation source 130 is rotated in the above manner, the radiation source 130 may be located in plural positions with parallaxes.

With this arrangement, one of plural images captured in different positions with parallaxes is visually recognized by the right eye, and the other one of the images is visually recognized by the left eye. In this manner, an image may be stereoscopically viewed.

A nut 147b of a ball screw 147 is attached to the box 146. A screw shaft 147a of the ball screw 147 is attached to a support pillar 148. As the screw shaft 147a is rotated by a motor (not shown), the nut 147b, the box 146, and the C-shaped arm 140 move up and down. By moving the C-shaped arm 140 up and down, the height of the center of rotation of the C-shaped arm 140 may be varied. The lower end of the support pillar 148 is attached onto a pillar supporting member 152 that horizontally protrudes from near a lower end portion of the housing of the main body 150.

Wheels 154 are attached to the bottom of the main body 150, so that the radiation generator 34 may move around.

The main body 150 contains a communication interface unit 132, a source control unit 134, and a source drive control unit 136 that are described later.

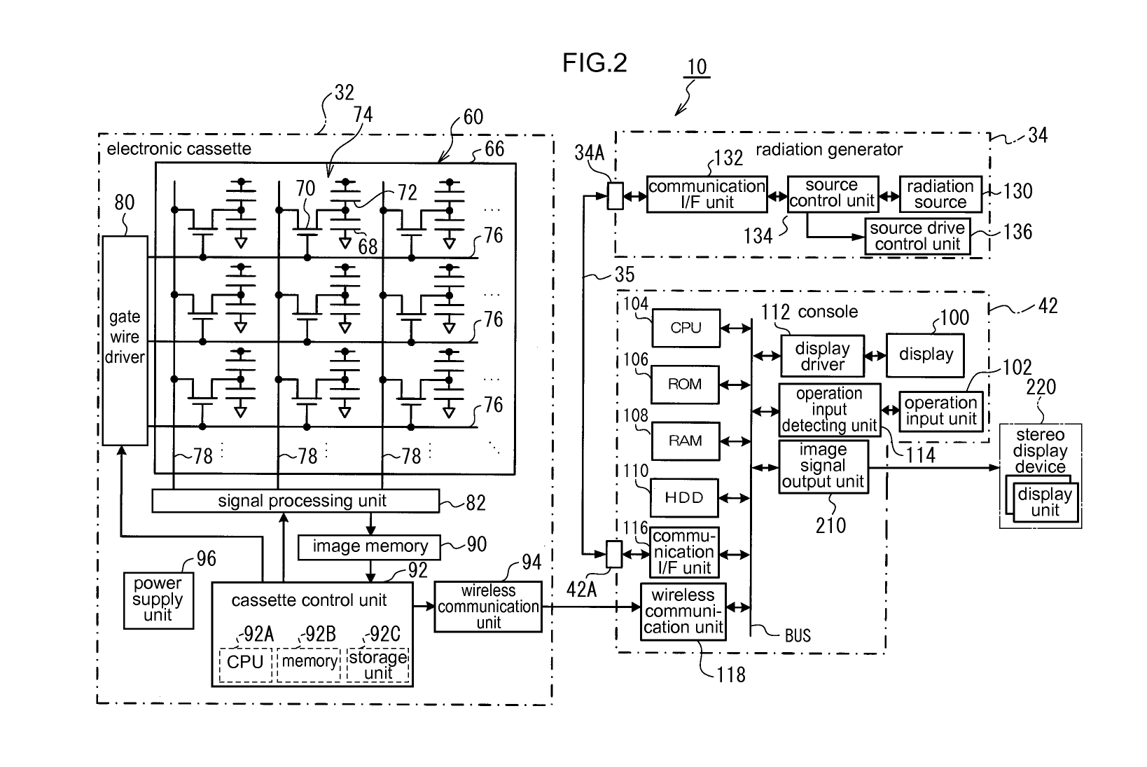

FIG. 2 is a block diagram showing the structure of the radiation image capturing apparatus 10 according to this exemplary embodiment.

The radiation generator 34 has a connecting terminal 34A for performing communication with the console 42. The console 42 has a connecting terminal 42A for performing communication with the radiation generator 34. The radiation generator 34 is connected to the console 42 via a communication cable 35.

A radiation detector 60 installed in the electronic cassette 32 is formed by stacking a photoelectric conversion layer on a TFT active-matrix substrate 66. The photoelectric conversion layer absorbs a radiation ray and convert the radiation ray into charges. The photoelectric conversion layer is made of amorphous selenium (a-Se) containing selenium as a main component (the content rate being 50% or higher, for example). When a radiation ray is applied to the photoelectric conversion layer, charges (pair of electron-hole) are internally generated in an amount equivalent to the level of the applied radiation. In this manner, the applied radiation ray is converted into charges. The radiation detector 60 may convert a radiation ray indirectly into charges by using a fluorescent material and a photoelectric conversion element (a photodiode), instead of the radiation-charge converting material such as amorphous selenium that converts a radiation ray directly into charges. As for the fluorescent material, gadolinium oxysulfide (GOS) and cesium iodide (CsI) are well known. In this case, a radiation-light conversion is performed with the fluorescent material, and a light-charge conversion is performed with the photodiode of the photoelectric conversion element.

A large number of pixel units 74 (the photoelectric conversion layer corresponding to the respective pixel units 74 being schematically shown as photoelectric conversion units 72 in FIG. 2) each including a storage capacitor 68 that stores charges generated from the photoelectric conversion layer and a TFT 70 for reading the charges stored in the storage capacitor 68 are arranged in a matrix fashion on the TFT active-matrix substrate 66. The charges generated in the photoelectric conversion layer as a result of radiation application to the electronic cassette 32 are stored in the storage capacitors 68 of the respective pixel units 74. With this arrangement, the image information carried by the radiation ray applied onto the electronic cassette 32 is converted into charge information, and is carried by the radiation detector 60.

Also, plural gate interconnects 76 and plural data interconnects 78 are provided on the TFT active-matrix substrate 66. The gate interconnects 76 extend in one direction (the row direction), and switch on and off the TFTs 70 of the respective pixel units 74. The data interconnects 76 extend in a direction perpendicular to the gate interconnects 78, and read the stored charges from the storage capacitors 68 via switched-on TFTs 70. The respective gate interconnects 76 are connected to a gate wire driver 80, and the respective data interconnects 78 are connected to a signal processing unit 82. When charges are stored in the storage capacitors 68 of the respective pixel units 74, the TFTs 70 of the respective pixel units 74 are sequentially switched on by the row by signals supplied from the gate wire driver 80 via the gate interconnects 76. The charges stored in the storage capacitors 68 of the pixel units 74 having the TFTs 70 switched on are transmitted as analog electrical signals through the data interconnects 78, and are then input to the signal processing unit 82. In this manner, the charges stored in the storage capacitor 68 of the respective pixel units 74 are sequentially read out by the row.

The signal processing unit 82 operates under the control of a cassette control unit 92 described later, and detects the amount of charges stored in the storage capacitors 68 of the respective pixel units 74 by the row. The signal processing unit 82 then outputs digital image information.

An image memory 90 is connected to the signal processing unit 82. Image information and error information that are output from the signal processing unit 82 are sequentially stored into the image memory 90. The image memory 90 has such a storage capacity as to store image information about a predetermined number of radiation images. Every time charges of one line are read out, the image information about the read one line is sequentially stored into the image memory 90.

The image memory 90 is connected to the cassette control unit 92 that controls operations of the entire electronic cassette 32. The cassette control unit 92 is realized by a microcomputer, and includes a CPU 92A, a memory 92B containing a ROM and a RAM, and a nonvolatile storage unit 92C formed by a HDD or a flash memory.

A wireless communication unit 94 is connected to the cassette control unit 92. The wireless communication unit 94 complies with wireless LAN (local area network) standards such as IEEE (Institute of Electrical and Electronics Engineers) 802.11a/b/g, and controls transmission of various kinds of information with external devices through wireless communication. The cassette control unit 92 may perform wireless communication with the console 42 via the wireless communication unit 94, and may exchange various kinds of information with the console 42. The cassette control unit 92 stores later described irradiation conditions received from the console 42, and, based on the irradiation conditions, starts the reading of charges.

A power supply unit 96 is also provided in the electronic cassette 32. The above described various circuits and elements (the gate wire driver 80, the signal processing unit 82, the image memory 90, the wireless communication unit 94, and the microcomputer functioning as the cassette control unit 92) are actuated by the power supplied from the power supply unit 96. The power supply unit 96 contains a battery (a rechargeable secondary cell) so as to maintain the portability of the electronic cassette 32, and supplies power from the charged battery to the various circuits and elements. In FIG. 2, the interconnects that connect the power supply unit 96 to the various circuits and elements are not shown.

The console 42 includes a display 100 that displays an operation menu, a captured radiation image, and the like, and an operation input unit 102 that is designed to have plural keys and has various kinds of information and operation instructions input therethrough.

The console 42 further includes: a CPU 104 that controls operations of the entire apparatus; a ROM 106 in which various kinds of programs including a control program are stored in advance; a RAM 108 that temporarily stores various kinds of data; a HDD 110 that stores and holds various kinds of data; a display driver 112 that controls displaying of various kinds of information on the display 100, and receives operation information from the display 100; an operation input detecting unit 114 that detects an operation state of the operation input unit 102; an image signal output unit 210 that outputs image signals to the stereo display device 220; a communication interface unit 116 that is connected to the connecting terminal 42A, and exchanges various kinds of information, such as the irradiation conditions, imaging site information, and the status information about the radiation generator 34, with the radiation generator 34 via the connecting terminal 42A and the communication cable 35; and a wireless communication unit 118 that exchanges various kinds of information such as the irradiation conditions and image information with the electronic cassette 32 through wireless communication.

The CPU 104, the ROM 106, the RAM 108, the HDD 110, the display driver 112, the operation input detecting unit 114, the image signal output unit 210, the communication interface unit 116, and the wireless communication unit 118 are connected to one another via a system bus BUS. Therefore, the CPU 104 may access the ROM 106, the RAM 108, and the HDD 110. Also, the CPU 104 may control the displaying of various kinds of information on the display 100 via the display driver 112, recognize the operation information from the display 100, control the image to be displayed on the stereo display device 220 via the image signal output unit 210, control the exchange of various kinds of information with the radiation generator 34 via the communication interface unit 116, and control the exchange of various kinds of information with the electronic cassette 32 via the wireless communication unit 118. Further, the CPU 104 may recognize the user operation state of the operation input unit 102 via the operation input detecting unit 114.

The radiation generator 34 includes: the radiation source 130 that outputs a radiation ray; the communication interface unit 132 that exchanges various kinds of information, such as the irradiation conditions, the imaging site information, and the status information about the radiation generator 34, with the console 42; the source control unit 134 that controls the radiation source 130, based on the received irradiation conditions; and the source drive control unit 136 that controls operations of the ball screw 147 and the gear 145 by controlling the power supply to the motor (not shown) driving the ball screw 147 and the gear 145.

The source control unit 134 is also realized by a microcomputer, and stores the received irradiation conditions, imaging site information, and the like. The irradiation conditions received from the console 42 contain information such as tube voltage, tube current, and irradiation time. Based on the received irradiation conditions, imaging site information, and the like, the source control unit 134 controls the C-shaped arm 140 by controlling the motor (not shown) driving the gear 145. By doing so, the source control unit 134 adjusts the angle at which the X-ray 131 emitted from the radiation source 130 is incident on the cassette 32 and the object 50. In this manner, the source control unit 134 causes the radiation source 130 to emit the X-ray 131, based on the received irradiation conditions.

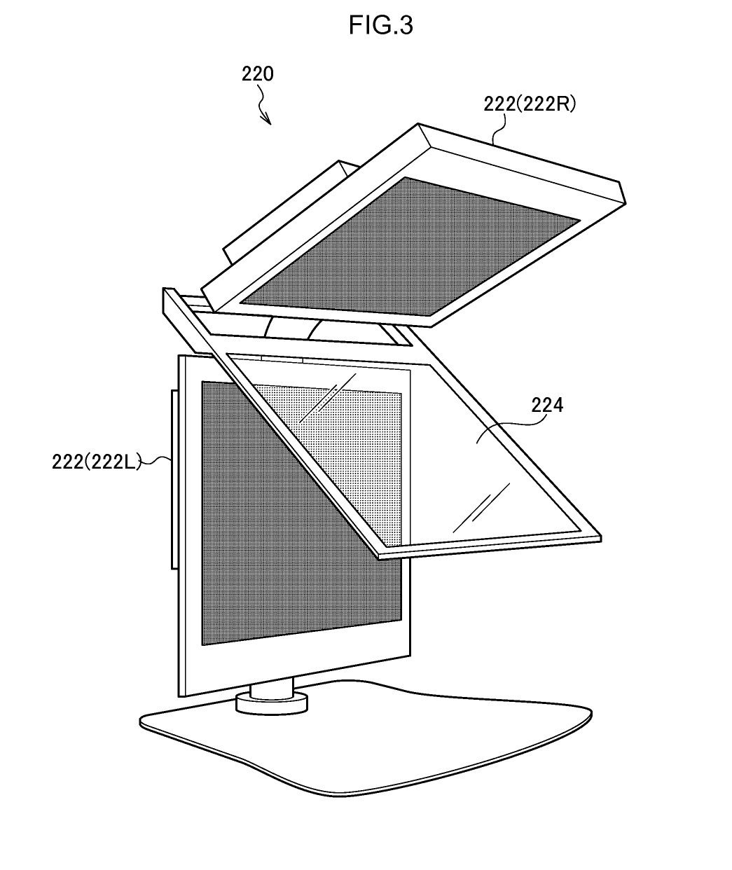

FIG. 3 illustrates an example structure of the stereo display device 220 according to this exemplary embodiment.

As shown in the drawing, in the stereo display device 220, two display units 222 are vertically arranged, and the upper display unit 222 is tilted forward and is fixed. The two display units 222 have display-light polarizing directions perpendicular to each other. The upper display unit 222 is a display unit 222R that displays an image for the right eye, and the lower display unit 222 is a display unit 222L that displays an image for the left eye. A beam splitter mirror 224 that transmits the display light emitted from the display unit 222L and reflects the display light emitted from the display unit 222R is provided between the display units 222L and 222R. The beam splitter mirror 224 is fixed at an angle that is adjusted so that the image displayed on the display unit 222L and the image displayed on the display unit 222R overlap with each other when an observer sees the stereo display device 220 from the front.

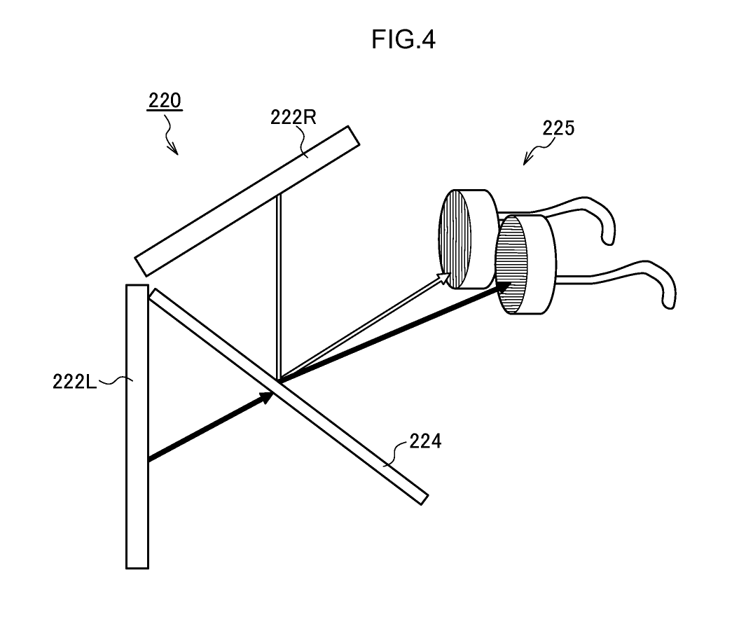

As shown in FIG. 4, by seeing the stereo display device 220 through polarizing glasses 225 formed by a right lens and a left lens that have polarizing directions perpendicular to each other, the observer may view the image displayed on the display unit 222L and the image displayed on the display unit 222R with the right eye and the left eye independently of each other. In this manner, the observer may stereoscopically view an image.

Next, the functions of the radiation image capturing apparatus 10 according to this exemplary embodiment are described.

When a radiation image is to be stereoscopically captured, the positional information about the radiation source 130, the information about the electronic cassette 32, the irradiation conditions, the imaging site information, and the like are input to the console 42 via the operation input unit 102 in the radiation image capturing apparatus 10.

The console 42 transmits the input positional information about the radiation source 130, the information about the electronic cassette 32, the exposure conditions such as tube voltage, tube current and irradiation time, the imaging site information, and the like to the radiation generator 34.

The console 42 also transmits image capturing control information, such as the irradiation time during which the radiation generator 34 keeps emitting a radiation ray when a radiation image is to be captured, to the electronic cassette 32 through wireless communication.

The radiation generator 34 adjusts the height of the C-shaped arm 140 so that the height of the center of rotation of the C-shaped arm 140 or the height of the center of rotation of the radiation source 130 becomes equal to the height of the upper surface 32a of the electronic cassette 32.

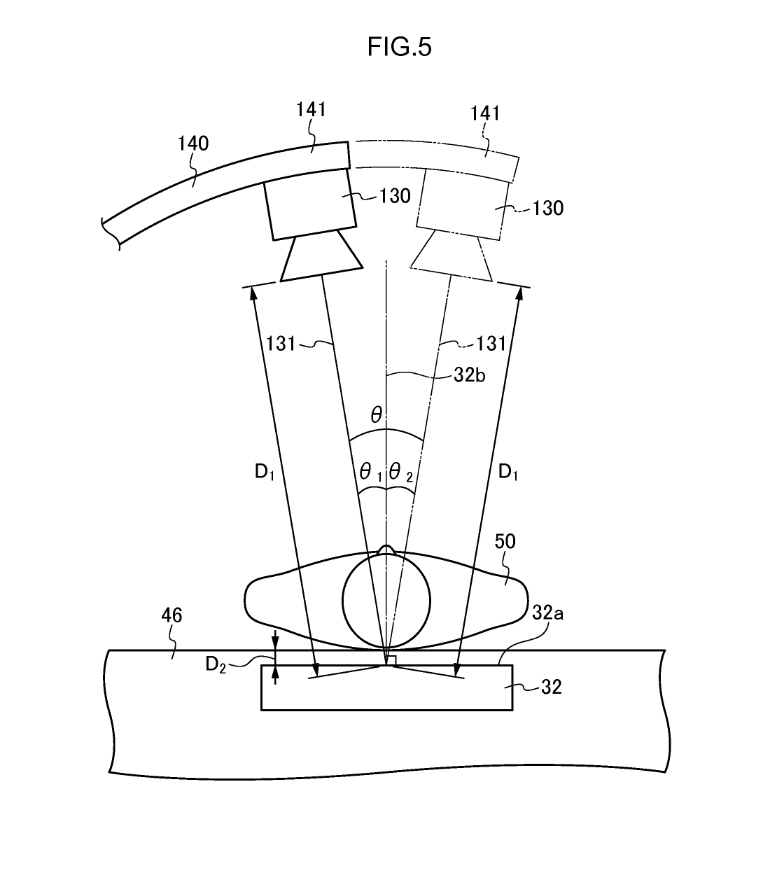

The radiation generator 34 then rotates the C-shaped arm 140, and positions the radiation source 130 at a predetermined angle θ1 with respect to a direction 32b perpendicular to the surface 32a of the electronic cassette 32, as shown in FIG. 5.

The radiation generator 34 then emits the X-ray 131 from the radiation source 130 under predetermined irradiation conditions. The X-ray 131 emitted from the radiation source 130 carries image information about the object 50 after transmitted through the object 50, and then reaches the electronic cassette 32 serving as a radiation detector.

The X-ray 131 carrying the image information is converted into an electrical signal by the electronic cassette 32, and the electrical signal is stored into the image memory 90.

After the image is captured, the cassette control unit 92 transmits the image information stored in the image memory 90 to the console 42 through wireless communication.

The console 42 performs various kinds of image corrections such as a shading correction on the received first image information, and stores the corrected first image information together with first image capturing information into the HDD 110. The first image capturing information contains the positional information about the radiation source 130 (such as the angle information (θ1) about the radiation source 130 and the distance D1 between the radiation source 130 and the electronic cassette 32), the information about the electronic cassette 32 (such as the distance D2 between the electronic cassette 32 and the object 50, the information as to whether the electronic cassette 32 has a holder, and the type of the holder if the electronic cassette 32 has one), the irradiation conditions such as tube voltage, tube current and irradiation time, the imaging site information and the like.

The electronic cassette 32 performs a reset operation to stand by for the next image capturing operation.

To capture a second image at a different parallax angle for stereoscopic viewing by changing the position of the radiation source 130, the positional information about the radiation source 130, the irradiation conditions, and the like are input to the console 42 via the operation input unit 102. In many cases, the irradiation conditions and the like for the second image are the same as those for the first image.

The console 42 transmits the positional information about the radiation source 130, the exposure conditions such as tube voltage, tube current, and irradiation time, and the like to the radiation generator 34.

The console 42 also transmits image capturing control information, such as the irradiation time during which the radiation generator 34 keeps emitting a radiation ray when a radiation image is to be captured, to the electronic cassette 32 through wireless communication.

In the case of the second image, the height of the center of rotation of the C-shaped arm 140, or the height of the center of rotation of the radiation source 130 is the same as the height in the case of the first image.

The radiation generator 34 then rotates the C-shaped arm 140, and positions the radiation source 130 at a predetermined angle θ2 with respect to the direction 32b perpendicular to the surface 32a of the electronic cassette 32 (or at a parallax angle θ (=θ1+θ2) with respect to the angle in the case of the first image capturing), as shown in FIG. 5. The distance Dl between the radiation source 130 and the electronic cassette 32 is maintained.

The radiation generator 34 then emits the X-ray 131 from the radiation source 130 under predetermined irradiation conditions. The X-ray 131 emitted from the radiation source 130 carries image information about the object 50 after transmitted through the object 50, and then reaches the electronic cassette 32 serving as a radiation detector.

The X-ray 131 carrying the image information is converted into an electrical signal by the electronic cassette 32, and the electrical signal is stored into the image memory 90.

After the image is captured, the cassette control unit 92 transmits the image information stored in the image memory 90 to the console 42 through wireless communication.

The console 42 performs various kinds of image corrections such as a shading correction on the received second image information, and stores the corrected second image information together with second image capturing information into the HDD 110. The second image capturing information contains the positional information about the radiation source 130 (such as the angle information (θ1) about the radiation source 130 and the distance D1 between the radiation source 130 and the electronic cassette 32), the information about the electronic cassette 32 (such as the distance D2 between the electronic cassette 32 and the object 50, the information as to whether the electronic cassette 32 has a holder, and the type of the holder if the electronic cassette 32 has one), the irradiation conditions such as tube voltage, tube current and irradiation time, the imaging site information, and the like.

At this point, the second image information and image capturing information are stored, together with the first image information and image capturing information, and the parallax difference (θ=θ1+θ2) in the first and second image capturing operations, into the HDD 110. The information is stored as the image information and image capturing information about two stereoscopic viewing images obtained by one image capturing operation.

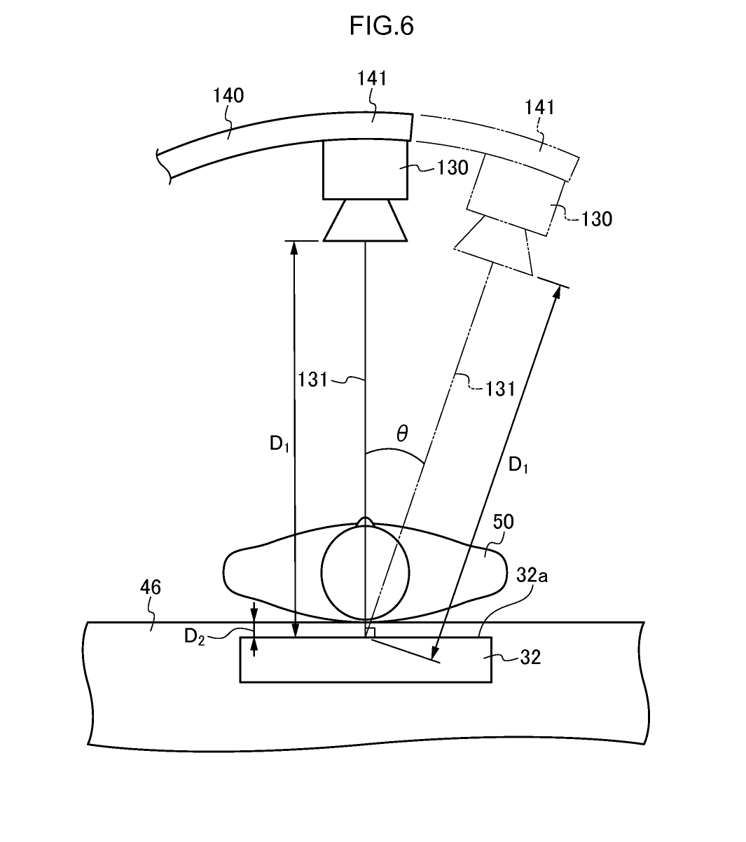

Also, as shown in FIG. 6, the first radiation image (a perpendicular image) may be captured from a direction perpendicular to the surface 32a of the electronic cassette 32, and the C-shaped arm 140 is then rotated so that the radiation source 130 is positioned at the predetermined angle θ with respect to the direction 32b perpendicular to the surface 32a of the electronic cassette 32 (or at the same parallax angle θ as in the first image capturing operation). The second radiation image may be then captured. Alternatively, the first image may be captured while the radiation source 130 is positioned at the predetermined angle θ with respect to the direction 32b perpendicular to the surface 32a of the electronic cassette 32, and the second image may be captured from a direction perpendicular to the surface 32a of the electronic cassette 32.

In such a case, the first image information and image capturing information, the second image information and image capturing information, the parallax difference (θ) in the first and second image capturing operations, and the information as to which one of the first and second images is a perpendicular image are stored as the image information and image capturing information about the two stereoscopic viewing images obtained through one image capturing operation, into the HDD 110.

The following is a description of a stereo image forming operation to be performed by the console 42 to cause the stereo display device 220 to display a stereo image based on the two radiation images stored as one piece of image capturing information in the HDD 110.

When a predetermined stereo image display start instruction is issued to the operation input unit 102, the console 42 performs the stereo image forming operation to form an image for the right eye and an image for the left eye that may be stereoscopically viewed, and causes the stereo display device 220 to display a stereo image.

The program for the stereo image forming operation is stored beforehand in a predetermined region in the ROM 106, and is executed by the CPU 104.

The program for the stereo image forming operation is performed to generate three-dimensional information based on the two stored radiation images, form the image for the right eye and the image for the left eye, cause the display unit 222R to display the image for the right eye, and cause the display unit 222L to display the image for the left eye. At this point, the image for the right eye and the image for the left eye are positioned, with a predetermined amount of offset being kept in the horizontal direction.

With this arrangement, an observer such as a physician may stereoscopically interpret radiation images and make a diagnosis from radiation images by viewing the screen of the stereo display device 220 through the polarizing glasses 225.

The observer such as a physician inputs information as to which image of the two images was used as a diagnosis confirmation image via the operation input unit 102 or the display 100. The information is stored as observation information related to the information about the two stereoscopic viewing images obtained through one image capturing operation, into the HDD 110. The amount of offset in the horizontal direction between the image for the right eye and the image for the left eye is also stored into the HDD 110. The amount of offset is stored as observation information related to the information about the two stereoscopic viewing images obtained through one image capturing direction.

In the above described example, stereoscopic viewing is performed with the polarizing glasses 225. However, stereoscopic viewing using glasses may be performed in a different manner. Further, glasses-free stereoscopic viewing with the use of a lenticular or the like may be performed with the naked eye. Therefore, the information as to which one of the stereoscopic viewing techniques is used, as well as the above described information, is stored as the observation information about the two stereoscopic viewing images obtained through one image capturing operation, into the HDD 110.

As described above, in this exemplary embodiment, plural images of the object 50 captured from different angles are associated with the stereoscopic viewing conditions information about stereoscopic viewing performed with the use of plural images, and are then stored into the HDD 110. Therefore, the stereoscopic viewing performed in diagnosing may be readily reproduced.

The stereoscopic viewing conditions information contains information about a stereoscopic viewing technique, or contains information as to whether a technique involving glasses is used, information as to which technique involving glasses is used, and information as to whether stereoscopic viewing is performed with the naked eye.

The stereoscopic viewing conditions information also contains the amount of horizontal offset between plural images. The degree of protrusion in stereoscopic viewing is determined by the parallax difference at the time of image capturing. However, the positions of protrusions in the depth direction are determined by the amount of horizontal offset in the stereoscopic viewing. Therefore, by recording the amounts of offset, the directions of protrusions in the stereoscopic viewing may be reproduced.

Also, information as to which one of the plural images is the diagnosis confirmation image is associated with the images obtained by the stereoscopic viewing, and is stored into the HDD 110. Accordingly, it is easy to determine which image is used as the diagnosis confirmation image.

Further, information as to which one of the plural images is a perpendicular image is associated with the images obtained by the stereoscopic viewing, and is stored into the HDD 110. Accordingly, it is easy to determine which image is the perpendicular image, and a diagnosis may be made based on the same image as a regular radiation image that is not stereoscopically viewed.

Also, information about the image capturing conditions under which the plural images are captured is associated with the plural images, and is stored into the HDD 110. Therefore, plural images for stereoscopic viewing may be readily captured under the same conditions in the future. The information about the image capturing conditions is useful in medical follow-ups.

The image capturing conditions information contains the distance between the radiation source 130 and the electronic cassette 32 serving as the radiation detector, and the parallax angle (θ) used when the plural images are captured.

The image capturing conditions information also contains the distance between the object 50 and the electronic cassette 32 serving as the radiation detector.

The image capturing conditions information further contains holder information about the electronic cassette 32 serving as the radiation detector, or information as to whether there is a holder and information as to the type of the holder or the like.

In the above described exemplary embodiment, the portable electronic cassette 32 is used as a radiation detector. However, instead of the electronic cassette 32, a stationary radiation detector may be used.

In the above described exemplary embodiment, an X-ray is used as a radiation ray. However, the invention is not limited to X-rays, and a γ-ray or the like may be used, instead of an X-ray, for example.

Various exemplary embodiments of the invention have hitherto been described, however, the invention is not limited to the exemplary embodiments. Therefore, the scope of the invention is limited only by the appended claims.

What is claimed is:

1. A radiation image processing device comprising:
a storage unit; and
a control unit that performs a control operation to associate a plurality of images of an object captured with radiation from different angles, with information about stereoscopic viewing conditions under which stereoscopic viewing has been performed with the plurality of images, the plurality of images associated with the information being stored into the storage unit through the control operation.

2. The radiation image processing device of claim 1, wherein the control unit performs a control operation to associate the plurality of images with information about image capturing conditions under which the plurality of images have been captured, the plurality of images associated with the information being stored into the storage unit through the control operation.

3. The radiation image processing device of claim 1, wherein the information about the stereoscopic viewing conditions includes a stereoscopic viewing technique.

4. The radiation image processing device of claim 1, wherein the information about the stereoscopic viewing conditions includes an amount of offset between the plurality of images.

5. The radiation image processing device of claim 2, wherein the information about the image capturing conditions includes a distance between a radiation source and a radiation detector, and a parallax angle used in capturing the plurality of images.

6. The radiation image processing device of claim 2, wherein the information about the image capturing conditions includes a distance between the object and a radiation detector.

7. The radiation image processing device of claim 2, wherein the information about the image capturing conditions includes holder information about the radiation detector.

8. The radiation image processing device of claim 1, wherein the control unit performs a control operation to store information as to which one of the plurality of images is a diagnosis confirmation image, into the storage unit.

9. The radiation image processing device of claim 1, wherein the control unit performs a control operation to store information as to which one of the plurality of images is a perpendicular image.

10. A radiation image processing method comprising storing a plurality of images of an object captured with radiation from different angles into a storage unit under a control of a control unit, the plurality of images being associated with information about stereoscopic viewing conditions under which stereoscopic viewing has been performed with the plurality of images.

11. The radiation image processing method of claim 10, further comprising storing the plurality of images into the storage unit under the control of the control unit, the plurality of images being associated with information about image capturing conditions under which the plurality of images have been are captured.

12. The radiation image processing method of claim 10, wherein the information about the stereoscopic viewing conditions includes a stereoscopic viewing technique.

13. The radiation image processing method of claim 10, wherein the information about the stereoscopic viewing conditions includes an amount of offset between the plurality of images.

14. The radiation image processing method of claim 11, wherein the information about the image capturing conditions includes a distance between a radiation source and a radiation detector, and a parallax angle used in capturing the plurality of images.

15. The radiation image processing method of claim 11, wherein the information about the image capturing conditions includes a distance between the object and a radiation detector.

16. The radiation image processing method of claim 11, wherein the information about the image capturing conditions includes holder information about the radiation detector.

17. The radiation image processing method of claim 10, further comprising storing information as to which one of the plurality of images is a diagnosis confirmation image into the storage unit under the control of the control unit.

18. The radiation image processing method of claim 10, further comprising storing information as to which one of the plurality of images is a perpendicular image into the storage unit under the control of the control unit.

19. A non-transitory computer-readable medium storing a program that causes a computer to perform a process including storing a plurality of images of an object captured with radiation from different angles into a storage unit under a control of a control unit, the plurality of images being associated with information about stereoscopic viewing conditions under which stereoscopic viewing has been performed with the plurality of images.
